(19) United States
(12) Patent Application Publication
(10) Pub. No.: US 2023/0381441 A1
(43) Pub. Date: Nov. 30, 2023

(54) NEONATAL AND PEDIATRIC NASAL CANNULA

(71) Applicants: DAVID KOHAN-GHADOSH, LOS ANGELES, CA (US); WADSWORTH WILLIAMS, Los Angeles, CA (US)

(72) Inventors: DAVID KOHAN-GHADOSH, LOS ANGELES, CA (US); WADSWORTH WILLIAMS, Los Angeles, CA (US)

(21) Appl. No.: 18/323,034

(22) Filed: May 24, 2023

Related U.S. Application Data
(60) Provisional application No. 63/345,157, filed on May 24, 2022.

(51) Int. Cl. A61M 16/06

(52) U.S. Cl. CPC A61M 16/0672; A61M 2240/00; A61M 2205/02

(57) ABSTRACT

A nasal cannula for kids and infants. The nasal cannula includes a reservoir tube and two inhalation members that extend from the reservoir tubing. The two inhalation members are configured to be inserted into two nostrils of a patient. Each of the inhalation members is integral with the reservoir tube and has an opening for delivering oxygen. Each inhalation member has a lower portion, a neck, and an upper portion. The lower portion forms a conical frustum shape that plugs a nostril, and the upper portion is of a bead shape that anchors within the nostril.

### CROSS-REFERENCE TO RELATED APPLICATIONS

This application claims priority from a U.S. Provisional Patent Application Ser. No. 63/345,157, filed on May 24, 2022, the disclosure of which is incorporated herein by reference in their entirety.

### FIELD OF INVENTION

The present invention relates to neonatal and pediatric nasal cannula, and more particularly, the present invention relates to improvements in neonatal and pediatric nasal cannula that have ventilation cutouts.

### BACKGROUND

Neonatal and pediatric nasal cannulas are available for infants and kids for providing supplemental oxygen. Chiefly, the neonatal and pediatric nasal cannulas, currently available, are miniaturized versions of the adult version. The use of such cannulas presents several problems for both the healthcare professional and patients. Three key shortcomings of nasal cannulas for infants are oxygen leak, the cannula falling out of place, and irritation from the adhesives used to try to keep the cannula in place. The oxygen leak makes it exceedingly difficult for physicians to titrate the correct amount of oxygen for fragile neonates. Too little oxygen can result in developmental delays or even death. Too much oxygen, however, can result in chronic lung disease and blindness. The cannula falling out of place results in not enough oxygen being delivered, leading to alarms, care teams having to rush in, and parents being rightfully worried. The last problem is the irritation due to adhesive. Premature babies have very delicate skin, so the adhesives often cause damage when worn for long or being replaced.

Thus, a long and unsolved need is there for infant and pediatric nasal cannulas that are devoid of the aforesaid drawbacks with conventional infant and pediatric cannulas.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a nose cannula for infants and kids that is devoid of the drawbacks including oxygen leak, the cannula falling out of place, and irritation from the adhesives.

It is another object of the present invention that the nose cannula is easy to use in infants and kids.

It is still another object of the present invention that the nasal cannula is safe to use.

It is yet another object of the present invention that the nasal cannula provides effective delivery of oxygen.

It is a further objective of the present invention that the nasal cannula can increase positive end-expiratory pressure.

It is still a further object of the present invention that the nasal cannula allows a patient to exhale through the nose.

It is an additional object of the present invention that the nasal cannula permits bottle/breastfeeding.

In one aspect, disclosed is a nasal cannula comprising a reservoir tube; and a first inhalation member that extends from the reservoir tube and is configured to fit into a nostril of a patient, the first inhalation member has a proximal end and a distal end, the proximal end is integral with the reservoir tube, the distal end has an opening, the inhalation member is hollow and in fluid communication with the reservoir tube. The first inhalation member comprises a lower portion of a tapered profile, the lower portion tapers from a bottom towards a neck of the first inhalation member, and an upper portion extends between the opening and the neck, the upper portion is of a bead shape. The opening is offset from a longitudinal axis of the first inhalation member. The first inhalation member further comprises a plurality of ventilation cutouts in a wall of the first inhalation member. The plurality of ventilation cutouts are elongated and extends along a longitudinal axis of the first inhalation member. The nasal cannula further comprises a second inhalation member that extends from the reservoir tube and is spaced apart from the first inhalation member, the second inhalation member is identical to the first inhalation member.

In one aspect, disclosed is a method for providing oxygen to an infant or a kid, the method comprising providing a nasal cannula. The nasal cannula comprising a reservoir tube, and a first inhalation member that extends from the reservoir tube and is configured to fit into a nostril of a patient, the first inhalation member has a proximal end and a distal end, the proximal end is integral with the reservoir tube, the distal end has an opening, the inhalation member is hollow and in fluid communication with the reservoir tube. The first inhalation member comprises a lower portion of a tapered profile, the lower portion tapers from a bottom towards a neck of the first inhalation member, and an upper portion extends between the opening and the neck, the upper portion is of a bead shape. The method further comprises inserting the first inhalation member into the nostril of the patient, wherein the upper portion of the inhalation members anchors into the nostril securing the nasal cannula. The nasal cannula further comprises a second inhalation member that extends from the reservoir tube and spaced apart from the first inhalation member, the second inhalation member is identical to the first inhalation member, wherein the second inhalation member is inserted into a second nostril of the patient.

In one aspect, disclosed is an inhalation member for a nasal cannula, the inhalation member configured to fit into a nostril of a patient, the inhalation member comprising a lower portion of a tapered profile, the lower portion tapers from a bottom towards a neck of the inhalation member; and an upper portion extends from the neck towards a top of the inhalation member, the upper portion has an opening for delivering oxygen, the upper portion is of a bead shape. The opening is offset from a longitudinal axis of the inhalation member. The inhalation member further comprises a plurality of ventilation cutouts in a wall of the inhalation member. The plurality of ventilation cutouts are elongated and extends along a longitudinal axis of the inhalation member. The inhalation member is configured to couple to a reservoir tube of the nasal cannula.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a front view of the nasal cannula, according to an exemplary embodiment of the present invention.

FIG. 2 is a side view of the nasal cannula, according to an exemplary embodiment of the present invention.

FIG. 3 is a top view of the nasal cannula, according to an exemplary embodiment of the present invention.

FIG. 4 is a rear view of the nasal cannula, according to an exemplary embodiment of the present invention.

FIG. 5 is a perspective view of the nasal cannula, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, the reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting of embodiments of the invention. As used herein, the singular forms “a”, “an”, and “the” are intended to include the plural forms as well, unless the context indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed is a nasal cannula for infants and pediatrics that overcomes several problems in the conventional infant and pediatric nasal cannulas, primarily the problems of oxygen leak, the cannula falling out of place, and irritation from the adhesives. The disclosed cannula is specially adapted to the needs of kids and infants and allows them to have normal body movement while wearing the disclosed nasal cannula. The disclosed nasal cannula can conveniently fit into the nostril such that to increase the positive end-expiratory pressure. For the fit, the disclosed nasal cannula has a novel design that can be accommodated within the nostrils of a patient. Both shape and material allow the disclosed cannula to adapt to inner shape and volume of the nostril. By occupying the normal shape of the nostril, the cannula generates a small amount of pressure as air is exhaled, which is helpful for keeping the small airways of the infant open. Currently, this added pressure feature is not offered by conventional nasal cannula, and typically reserved for more invasive and expensive methods of supplemental oxygen therapy.

The ventilation cutouts in the disclosed nasal cannula allow the patient to exhale through the nose, while still generating positive end expiratory pressure because of the novel shape of the ventilation cannulas.

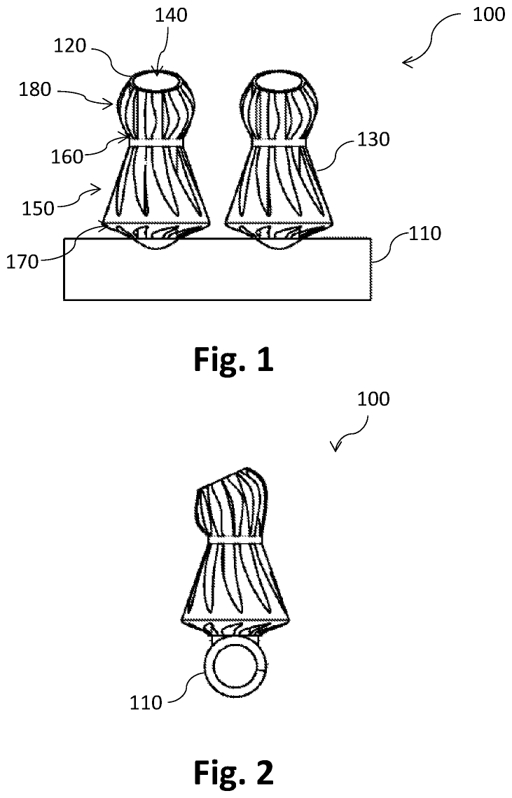

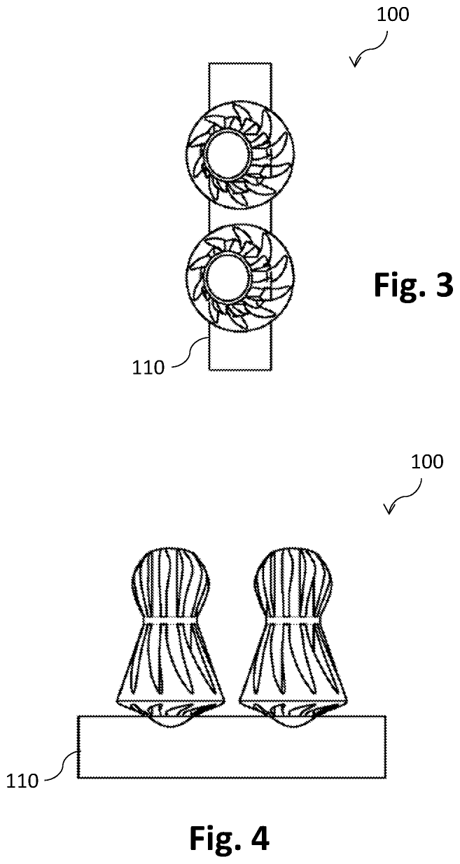

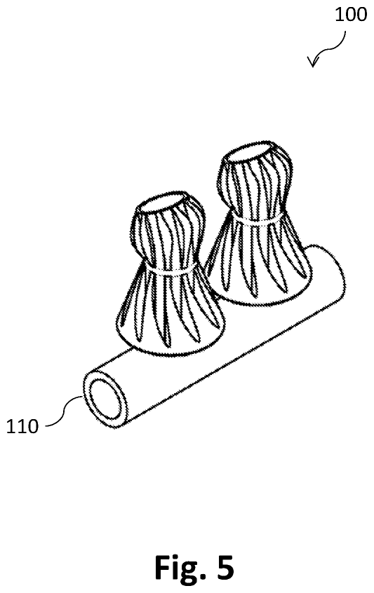

FIGS. 1-5 show different views of the disclosed nasal cannula 100 for infants and kids. The nasal cannula 100 can include a reservoir tube 110 that is of an elongate tubular profile. To the two ends of the reservoir tube 110, a headset with ear loops can be attached (not shown). The structure and functioning of the headset with ear loops are known in art. The headset with ear loops can further be connected to a cannula tubing which is also well-known in the art. The cannula tubing can be connected to an oxygen source. Any known headset with ear loops and cannula tubing with other functionalities known to a skilled person for use in the nasal cannula is within the scope of the present invention. Moreover, the ear loops can be optional, and the disclosed nasal cannula can be connected to the oxygen source through the cannula tubing. The size of the headset with ear loops and cannula tubing can be varied depending upon the age of the patient and any such variations is within the scope of the present invention.

A pair of inhalation members i.e., a first inhalation member 120 and a second inhalation member 130 extend from the reservoir tube 110. Each of the inhalation members is configured to be inserted into a nostril, wherein the first inhalation member and the second inhalation member are positioned closely together so that both can be inserted into the nostrils of the patient. The inhalation members, the reservoir tube, and the rest of the nasal cannula are in fluid communication such that oxygen from the oxygen source is carried through the cannula and ejects from the pair of inhalation members into the nose.

At least the inhalation members can be made of either silicon or thermoplastic polyurethane. This may allow for the members to be soft and compressible, so that the inhalation members can be compressed when inserted into a nostril and, upon insertion, can return to the normal shape and fit within the nostrils. The reservoir tube can also be made from silicon or thermoplastic polyurethane. It is understood, however, that any other material for the inhalation members and the reservoir tube is within the scope of the present invention. Preferably, the material should be soft and medical grade. Also, as shown in the drawings, the geometry of the inhalation members and the reservoir tube is so that the air pathway has an angle to it to direct the oxygen correctly.

The inhalation member has a proximal end and a distal end. The proximal end integrates with the reservoir tube and the distal end has an opening 140 for the oxygen to pass through. The inhalation member has a lower portion 150 that extends from the proximal end towards the middle of the inhalation member. As shown in the drawings, the lower portion is broad at the bottom and tapers inwards up to the narrow middle portion of the inhalation member. The middle portion is also referred to herein as a neck 160 of the inhalation member. The lower portion acts as a plug that allows for the nostrils to be occluded, as a result the oxygen leak is significantly reduced and also the positive end-expiratory pressure is increased which helps to keep the patient's airways open. The tapered portion resembles a frustoconical shape, and the neck of the inhalation member is narrow. The base 170 of the inhalation member at the proximal end can be conical in shape, wherein the tip of the cone is integral to the reservoir tube.

The upper portion 180 of the inhalation member extends between the distal end and the neck of the inhalation member. As shown in the drawings, the upper portion can be of a bead shape. The bead-shaped upper portion acts as an anchor for the nasal cannula, wherein the bead-shaped upper portion can anchor into the nostril for securing the nasal cannula. The distal end opening in the inhalation member is angled for improving oxygen delivery into the nasal cavity. The opening is offset from the longitudinal axis of the inhalation member. The opening 140 may be offset by about 20-30 degrees from the longitudinal axis. Preferably, the opening 140 can be offset by 24 to 25 degrees.

In certain implementations, the opening 140 can be about 4-5 mm wide. The maximum height of the inhalation member from the reservoir tube can be about 13-14 mm. The minimum height of the inhalation member, due to slanted opening, can be about 11-12 mm. Width of the neck can be about 0.5 to 1.0 mm. The distance between central longitudinal axis of the two inhalation members can be about 10 mm. The maximum width of the lower portion, also of the inhalation member, can be about 8-9 mm. The height of the base portion above the reservoir tube can be about 1-2 mm. The thickness of the reservoir tube can be about 5-6 mm. It is understood that the above measurements are for illustration purposes only, and the actual measurements may vary depending upon different factors, such as age of a patient.

The inhalation member may further include multiple ventilation cutouts 190 disposed throughout an area of the inhalation member. Through these ventilation cutouts, patients can exhale through their nose, thus helping to maintain the current benefits of the nasal cannula and also permitting bottle/breast-feeding and more movement of the baby. The ventilation cutouts can be of elongated and swirl profile that extends along the longitudinal axis of the inhalation member, such a profile of the ventilation cutouts in the wall of the inhalation member offers the least resistance to the exhaled air. Ventilation cutouts can also be provided in the base portion 170 of the inhalation member.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A nasal cannula comprising:
a reservoir tube; and
a first inhalation member that extends from the reservoir tube and is configured to fit into a nostril of a patient, the first inhalation member has a proximal end and a distal end, the proximal end is integral with the reservoir tube, the distal end has an opening, the inhalation member is hollow and in fluid communication with the reservoir tube, the first inhalation member comprises:
a lower portion of a tapered profile, the lower portion tapers from a bottom towards a neck of the first inhalation member, and
an upper portion extends between the opening and the neck, the upper portion is of a bead shape.

2. The nasal cannula of claim 1, wherein the opening is offset from a longitudinal axis of the first inhalation member.

3. The nasal cannula of claim 1, wherein the first inhalation member further comprises:
a plurality of ventilation cutouts in a wall of the first inhalation member.

4. The nasal cannula of claim 3, wherein the plurality of ventilation cutouts are elongated and extends along a longitudinal axis of the first inhalation member.

5. The nasal cannula of claim 1, wherein the nasal cannula further comprises:
a second inhalation member that extends from the reservoir tube and is spaced apart from the first inhalation member, the second inhalation member is identical to the first inhalation member.

6. The nasal cannula of claim 1, wherein the first inhalation member is made of silicon.

7. A method for providing oxygen to an infant or a kid, the method comprising:
providing a nasal cannula comprising:
a reservoir tube, and
a first inhalation member that extends from the reservoir tube and is configured to fit into a nostril of a patient, the first inhalation member has a proximal end and a distal end, the proximal end is integral with the reservoir tube, the distal end has an opening, the inhalation member is hollow and in fluid communication with the reservoir tube, the first inhalation member comprises:
a lower portion of a tapered profile, the lower portion tapers from a bottom towards a neck of the first inhalation member, and
an upper portion extends between the opening and the neck, the upper portion is of a bead shape; and
inserting the first inhalation member into the nostril of the patient, wherein the upper portion of the inhalation members anchors into the nostril securing the nasal cannula.

8. The method of claim 7, wherein the opening is offset from a longitudinal axis of the first inhalation member.

9. The method of claim 7, wherein the first inhalation member further comprises:
a plurality of ventilation cutouts in a wall of the first inhalation member.

10. The method of claim 7, wherein the plurality of ventilation cutouts are elongated and extends along a longitudinal axis of the first inhalation member.

11. The method to claim 7, wherein the nasal cannula further comprises:
a second inhalation member that extends from the reservoir tube and spaced apart from the first inhalation member, the second inhalation member is identical to the first inhalation member, wherein the second inhalation member is inserted into a second nostril of the patient.

12. The method of claim 7, wherein the first inhalation member is made of silicon.

13. An inhalation member for a nasal cannula, the inhalation member configured to fit into a nostril of a patient, the inhalation member comprising:
a lower portion of a tapered profile, the lower portion tapers from a bottom towards a neck of the inhalation member; and
an upper portion extends from the neck towards a top of the inhalation member, the upper portion has an opening for delivering oxygen, the upper portion is of a bead shape.

14. The inhalation member of claim 13, wherein the opening is offset from a longitudinal axis of the inhalation member.

15. The inhalation member of claim 13, wherein the inhalation member further comprises:
a plurality of ventilation cutouts in a wall of the inhalation member.

16. The inhalation member of claim 15, wherein the plurality of ventilation cutouts are elongated and extends along a longitudinal axis of the inhalation member.

17. The inhalation member of claim 13, wherein the inhalation member is configured to couple to a reservoir tube of the nasal cannula.
The inhalation member of claim 13, wherein the inhalation member is made of silicon.
